(19) United States
(12) Patent Application Publication
(10) Pub. No.: US 2007/0281034 A1
(43) Pub. Date: Dec. 6, 2007

(54) Magnetic microparticles comprising organic substances

(76) Inventors: Dmitri B. Kirpotin, San Francisco, CA (US); Daniel C.F. Chan, Denver, CO (US); Paul A. Bunn JR., Evergreen, CO (US)

Correspondence Address: Dmitri B. Kirpotin, PhD, Apt. 102, 435 43 Ave., San Francisco, CA, 94121, US

(21) Appl. No.: 11/803,902

(22) Filed: May 15, 2007

Related U.S. Application Data
(60) Provisional application No. 60/800,380, filed on May 15, 2006.

(51) Int. Cl. A61K 33/26; A61K 31/7048; A61K 31/704; A61K 31/525; A61K 9/14

(52) U.S. Cl. 424/489; 424/646; 514/34; 514/251; 514/28; 977/906

(57) ABSTRACT

The invention provides for water-dispersible microparticles comprising magnetic material and organic substance, such as a pharmaceutical or a pigment, that constitute in totality at least 50%, or preferably more that 70%, of the dry mass of the particle. The method of making such microparticles is also provided, comprising coating of the magnetic material in an aqueous colloid form with a layer of a polyelectrolyte, and combining the coated colloid with the organic substance having ionic charge opposite to that of the polyelectrolyte. When the organic substance is poorly soluble in water, the combining comprises dissolving of the substance in a water-miscible organic solvent, and combining said solution with the polyelectrolyte-coated magnetic colloid. The magnetic pharmaceutical microparticles are useful for magnetically-guided drug delivery, and/or for non-invasive monitoring of the drug distribution in the body. Magnetic microparticles comprising dyes and pigments are useful in magnetic inks.

### CROSS-REFERENCE TO RELATED APPLICATIONS

This Application claims benefit of priority of the U.S. Provisional Patent Application No. 60/800,380 filed on May 15, 2006, which is incorporated herein by reference in its entirety for all purposes.

### STATEMENT OF THE RIGHTS TO INVENTIONS MADE UNDER FEDERALLY SPONSORED RESEARCH AND DEVELOPMENT

This invention was supported, in part, by the National Institutes of Health, National Cancer Institute grant P50CA58187. The Government of the United States has certain rights in this invention.

### FIELD OR THE INVENTION

This invention pertains to the field of pharmaceuticals, magnetic drug carriers, and magnetic microparticles in general. The invention provides magnetic microparticles comprising pharmaceuticals as well as methods of making and using same.

### BACKGROUND OF THE INVENTION

There is a recognized need for pharmaceuticals having the ability to be confined in the selected anatomical site after systemic administration. One of the known methods to address this need is to endow a pharmaceutical with magnetic properties so that the confinement of the pharmaceutical in the desired anatomical site may be aided by a magnet. This is often done by combining a pharmaceutical and a magnetic component in a microscopic particle.

A variety of magnetic pharmaceuticals designed over the above principle have been previously reported. The pharmaceutical substance and the magnetic substance were co-incorporated into lipid vesicles (liposomes) (H. Kiwada, J. Sato, Y. Yamada, Y. Kato, 1986. Chem. Pharm. Bull. 34, 4253-4258.). The pharmaceutical substance and the magnetic component were co-incorporated into microspheres made of heat-denatured of chemically cross-linked protein (A. Senyei, C. Driscoll, K. J. Widder, 1985. Meth. Enzymol. 112, 56-67), chemically-crosslinked carbohydrate (M. I. Papissov, G. P. Samokhin, M. D. Smirnov, V. P. Torchilin, V. N. Smirnov, 1984. Bull. Exper. Biol. Med. (Moscow), 98, 372-374), or crystallized carbohydrate (U.S. Pat. No. 4,501,726). The pharmaceutical substance and the magnetic component were co-incorporated into a microscopic spheres made of cyanoacrylate polymer (A. Ibrahim, P. Couvreur, M. Roland, P. Speiser, 1983, J. Pharm. Pharmacol., 35, 59-61). The pharmaceutical substance and the magnetic component were co-dispersed in a viscous, biocompatible fluid (E. K. Ruuge, A. N. Rusetski, 1993. J. Magnetism Magnetic Mater. 122, 335-339). The pharmaceutical substance and the magnetic component were co-incorporated into a coacervation complex produced by two oppositely charged macromolecules (D. B. Kirpotin, A. F. Orlovski. In: Proc. 5th Internat. Conf. Magnetic Fluids, Salaspilss, Latvija, 1989, p. 260-261).

These previously known microparticulate magnetic pharmaceuticals, while sometimes forming stable dispersions in aqueous media, suffer from a common disadvantage of having a relatively low load of the pharmaceutical substance, and/or of a magnetic substance. This disadvantage is inherent to the art described above and is brought about by the need of having substantial amount of a neutral constituent to cement together the drug and the magnetic component in a magnetic pharmaceutical microparticle. Therefore, there is in the field an unmet need to provide for magnetic pharmaceutical carriers which are stably dispersible in aqueous medium, small in size, and comprise the bulk of their mass in the form of active ingredients, that is, the magnetic substance and the pharmaceutical compound. Similarly, in a technical field of coloring and printing substances, such as inks and paints, there is an unmet need to provide pigment and dye compositions in the magnetically susceptible form, for controlled disposition and/or detection of the images on various media.

### BRIEF SUMMARY OF THE INVENTION

The following invention is aimed to overcome the disadvantage of low magnetic and/or drug load in microparticulate magnetic carriers and therefore to increase the practical utility of magnetic pharmaceuticals. This is done by coating of a magnetic component with a substance having high affinity to a pharmaceutical, and also having such nature that the interaction between a pharmaceutical and such coated magnetic component in a solution will result in co-precipitation of the pharmaceutical and the magnetic component in the microparticulate form.

Specifically, we have coated the colloidal particles of magnetic iron oxide with a polyelectrolyte, and further let such polyelectrolyte-coated magnetic colloid to react, in a solution, with drug molecules capable of carrying ionic charge opposite to that of the coating. By way of theory, but without being limited by it, we hypothesize that the cooperative Coulombic forces between the drug molecules and the polyelectrolyte coating caused the complex formation between the drug and the magnetic component, and the reduced solubility of such complex further resulted into its separation from the solution in the form of microparticles suitable, for example, for oral or parenteral administration into the body for medical purposes.

Due to the relatively small amount of polyelectrolyte coating required for the above-described reaction, the resulting magnetic pharmaceutical microparticles are composed mostly of the drug and the magnetic substance, comprising together at least 50%, more often, 70%, and typically about 90% of the dry mass of a microsphere

It is understood that the same inventive considerations apply where organic dye and/or pigment substances are taken instead of pharmaceutical substances, to obtain coloring or printing compositions, such as, inks or paints, comprising water-dispersible magnetic dye or pigment microparticles with the similarly high summary content of the dye or pigment, and a magnetic material.

The invention includes a new material, and a method for its manufacturing and use.

In one embodiment, the invention provides for a composition comprising microparticles, said microparticles comprising:

(i) a magnetic substance;

(ii) a charged polymer associated with said magnetic substance, and having a charge sign; and

(iii) an ionizable organic compound,

wherein said ionizable organic compound in aqueous medium is ionizable to the charge sign opposite to that of said charged polymer, and wherein said magnetic substance and said organic compound in totality constitute at least 50% of the microparticle dry mass, and wherein said microparticles are dispersible in water. Typically, while a polyelectrolyte component of the magnetic microparticle is a polymer, the organic compound comprised in the microparticles is not a polymer (non-polymeric). Preferably, said magnetic substance and said organic compound in totality constitute at least 70%, or at least 90. of the microparticle dry mass. In certain embodiments, the magnetic substance is ferrite, magnetite, or maghemite. The microparticle is preferably between 10 nanometers and 10 micrometers in size, more preferably between 30 nanometers and 3 micrometers, and most preferably 1 micrometer or less. In one embodiment, the magnetic substance is in the form of a colloidal particle, nanoparticle, or nanocrystal, and comprises a single magnetic domain, or multiple domains. The magnetic substance is preferably a ferromagnetic, paramagnetic, or superparamagnetic substance, and is in some cases a nanoparticle having average size from about 1 nm to about 100 nm.

Further, in another embodiment, in addition, the organic compound constitutes at least 10% of the microparticle dry mass.

In yet another embodiment, the organic compound of the composition is poorly soluble in water, typically less than 2 mg/mL, and preferably 0.1 mg/mL or less.

In certain preferred embodiments, said organic compound is a pharmaceutical, such as an anticancer drug or an antibiotic drug.

In another embodiment, the charged polymer that coats the magnetic substance is a polymeric acid and said organic compound is ionizable to positive charge. Alternatively, the charged polymer is a polymeric base and said organic compound is ionizable to negative charge.

In another embodiment, the microparticle comprises a surface coating effective to extend the microparticle's circulation time. Preferably, said surface coating comprises a hydrophilic polymer, for example, whose chains ate terminally appended (grafted) on the particle surface. One such hydrophilic polymer comprises poly(alkyl ether), and is, in one embodiment, poly(ethylene glycol).

In yet another embodiment, said microparticle comprises a targeting moiety. The targeting moiety is a polypeptide, such as an antibody or an antigen-binding fragment thereof; a peptide, a nucleic acid, a polysaccharide, or a small molecule ligand, that forms a specific binding pair with the molecular marker present in the body area or cells of interest. The targeting moiety (ligand) may be linked directly to the microparticle of via a spacer, which is in one embodiment, said hydrophilic polymer.

The invention includes also a method of making a microparticle-containing composition, said method comprising the step of contacting a magnetic substance dispersed in a liquid medium and having a charged polymer associated with said magnetic substance and ionized in said medium to attain a charge sign, with an ionizable organic compound attaining in said medium a charge sign opposite to that of said charged polymer, to effect formation of a microparticle. The magnetic substance and said organic compound in totality constitute at least 50%, at least 70%, or at least 90%, of the microparticle dry mass. In some cases, said liquid medium comprises water (i.e. in an aqueous medium), and/or is essentially free of said charged polymer not associated with said magnetic substance.

In certain embodiments, the organic substance used in said method is poorly soluble in water. Then, said contacting comprises mixing of said magnetic substance dispersed in a water-containing medium with a solution of said organic substance in a medium comprising a water-miscible organic solvent.

In still another embodiment, the method of making a magnetic microparticle further comprises a step of coating said microparticle with a substance effective to prolong the microparticle's circulation time. In one case, said circulation-prolonging substance comprise, comprises a hydrophilic polymer. The coating then can be achieved, for example, contacting the microparticle with the hydrophilic polymer linked to a hydrophobic moiety. The hydrophilic polymer may further comprise a targeting moiety.

In still another embodiment, the pharmaceutical composition comprising an inventive microparticle containing a magnetic substance, a polyelectrolyte, and an organic compound, wherein said organic compound is a pharmaceutical and wherein said microparticles are in a pharmaceutically acceptable medium.

The invention also includes a method of treatment of a disease in a patient comprising administering to the patient an effective amount of the composition containing any of the microparticles described herein.

The invention further includes embodiments where said organic compound in the magnetic microparticle comprises a dye or a pigment, making together with the magnetic substance at least 50%, preferably at least 70%, and most preferably at least 90% of the particle dry mass, and compositions comprising such microparticles.

### BRIEF DESCRIPTION OF THE DRAWINGS

FIG. 1 illustrates the structure of an exemplary organic pharmaceutical compound doxorubicin (a), and exemplary polyelectrolyte chondroitinsulfate A (b), and a schematic representation of the formation of a microparticle III from the polyelectrolyte-coated magnetic substance I and the ionizable organic pharmaceutical compound, doxorubicin II.

FIG. 2 is a reproduction of a fluorescent microphotography image of microparticles containing doxorubicin and chondroitin sulfate A produced as described in Example 2 herein. Larger round bodies are mouse red blood cells added as a size reference.

FIG. 3 is a size distribution histogram obtained from a transmission electron microscopy image of gamma-ferric oxide magnetic nanoparticles coated with chondroitin sulfate A produced according to Example 1 herein.

FIG. 4A is a cell viability vs. drug concentration graph obtained in the culture of KB epidermoid carcinoma cells, comparing the effects of free (unbound) doxorubicin (open circles), and doxorubicin within magnetic microparticles prepared according to Examples 4 (filled circles) and 3 (triangles). Note that the magnitude of cell killing effect of these preparations is very close. DXR denotes doxorubicin.

FIG. 4B is a cell viability vs. drug concentration graph obtained in the culture of KB epidermoid carcinoma cells, comparing the effects of free (unbound) dexniguldipine (open circles), and dexniguldipine within magnetic microparticles prepared according to Example 5 (filled circles). Note that the magnitude of cell killing effect of these preparations is practically identical. DNG denotes dexniguldipine.

FIG. 4C is a cell viability vs. iron concentration graph obtained in the culture of KB epidermoid carcinoma cells, exposed to polyelectrolyte-coated magnetic iron oxide colloid prepared according to Example 1. Note that the magnetic colloid itself is not cytotoxic within the studied concentration range.

FIG. 5 is a cell viability vs. drug concentration graph obtained in the culture of NCI H-1048 lung carcinoma cells, comparing the effects of free (unbound) methotrexate (open circles), and methotrexate within magnetic microparticles prepared according to Example 6 (filled circles). Note that the magnitude of cell killing effect of the free drug and the drug in magnetic microparticles is practically identical. MTX denotes methotrexate.

### DETAILED DESCRIPTION OF THE INVENTION

The present invention relates to compositions comprising magnetic microparticles that include magnetic substance and organic substance having certain utility (also termed herein as organic active principle), such as a pharmaceutical substance, useful in treatment, diagnosis, or prevention or a disease, or a coloring substance, such as a pigment or a dye, useful in printing or otherwise creating images on various media. Combining of magnetic substance component and a pharmaceutical or coloring component in a microparticle is advantageous as it affords controllable disposition of the microparticle and its useful payload, the organic substance, by applying magnetic field, or modulation of the organic substance by applying high frequency electromagnetic field to generate heat within the particle, or using the magnetic properties of the magnetic component to quantify and follow the disposition of the organic compound, for example, in the patient body using magnetic imaging methods, such as MRI. The inventive particles show unexpectedly large summary payloads of a magnetic substance and the organic substance, and in a preferred embodiment, are also dispersible in water, surprisingly even when the organic substance itself is poorly water-soluble. The invention also relates to a method suitable for preparation of such microparticles and compositions.

The magnetic microparticle of the invention comprises a magnetic substance, preferably in a highly disperse (colloidal) form, a polyelectrolyte associated with (e.g., adsorbed on) the magnetic substance; and the organic substance, such as a drug, dye, or pigment, associated with such magnetic substance-polyelectrolyte in such proportions that the drug (or a dye or a pigment) and the magnetic substance together constitute at least 50%, at least 70%, or at least 90%, of the dry mass of the microparticle. To analyze the amount of the components in the particle dry mass, any methods known in the art are suitable. This include, without limitation, spectrophotometric analysis, chromatography methods (HPLC), mass-spectrometry, magnitometry, and induction-coupled plasma spectrometry for quantification of elements. If the components of the particles are known, and the separation of the particles from dissolved material is performed (e.g., by magnetic precipitation, as described in the examples herein), the quantification of magnetic substance, the polyelectrolyte, and the organic compound will be sufficient to perform calculations. As understood herein, a “dry mass” refers to the sum of the microparticle nonvolatile components, i.e., other than water and/or another solvent, and may, but does not have to, entail actual removal of water and/or other solvents and volatiles from the microparticle in order to be accurately assessed.

The magnetic substance of the microparticle is, for example, magnetite (Fe₃O₄), gamma-iron(III) oxide (maghemite), or a combination thereof; ferrite of a divalent metal(s), optionally containing lantanides or other metals of ferrous group (cobalt, nickel); or a magnetic metal in pure form or in a form or an alloy. The magnetic substance is preferably in the form of a colloid, nanoparticles, or nanocrystals, such as having sizes from about 1 nm to about 200 nm, preferably between 2-100 nm. By their physical properties, the magnetic substance in the form of a nanoparticle or nanocrystal may be ferromagnetic, paramagnetic, or superparamagnetic, and may constitute a single magnetic domain, or more than a single domain.

The magnetic substances may be prepared by any method known in the art. For example, iron oxide magnetic particles of ferrite, magnetite, maghemite (gamma-iron(III) oxide), as well as intermediate forms, can be prepared by alkaline precipitation from aqueous-solutions of ferric and ferrous salts according to the methods well known in the art. See, for example, U.S. Pat. No. 4,452,773; U.S. Pat. No. 5,916,539; U.S. Pat. No. 5,427,767. Particularly preferred methods include highly magnetic iron oxide nanoparticles according to U.S. Pat. No. 5,411,730, the teachings of which are incorporated herein by reference. In one exemplary method, to obtain nanoparticles of magnetite suitable to practice the invention, one would combine in an aqueous solution equimolar amounts of ferric and ferrous chloride and titrate the solution with concentrated ammonia, optionally containing a stabilizing polymer, such as dextran, or a polyelectrolyte as described herein, until pH 8-10 is achieved, and incubate the resulting slurry at temperatures of 50-70° C. for 5-30 min. Then, the reaction mixture is neutralized, for example, with acetic acid, and dialyzed to remove salts. The magnetic iron oxide can be then separated by a magnet and redispersed in distilled water, optionally—if no stabilizing polymer or polyelectrolyte is present—with small amounts of HCl and short boiling to achieve transition of the precipitate into colloidal state.

Polyelectrolytes suitable for the purpose of the invention are, in general, polymer molecules, that is, molecules consisting of repetitive units, preferably of similar chemical structure, with molecular weights, roughly defined, from 400 to 2,000,000, soluble in water, and containing in their structure ionizable groups, that is, chemical functional groups capable of electrolytic dissociation resulting in the formation of ionic charge. Examples of such ionizable groups are given above in the characterization of pharmaceuticals. According to the invention, net charge of the polyelectrolyte used to coat the magnetic substance is preferably the opposite to that of the pharmaceutical under the conditions at which the polyelectrolyte-coated magnetic substance and the pharmaceutical are combined.

The following list gives examples of such polyelectrolytes.

Acidic and basic polysaccharides—natural and derived from natural:

polygalacturonates, hyaluronic acid, gum arabic, chondroitin sulfates A, B, and C, keratan sulfates, dermatan sulfates, heparin and its derivatives, pectin and its derivatives, alginic (poly-anhydromannuronic) acid, teichoic acids, chitosans; derivatives of cellulose, amylose, amylopectin, dextran, or other neutral polysaccharide obtained by introduction of carboxyalkyl, phosphate, sulfate, amino-, mono-, di-, trialkylamino, tetraalkylammonium functional groups, derivatives of the said polysaccharides with nitrogen heterocycles, and derivatives obtained by grafting other ionizable functions to polysaccharide backbone.

Acidic and basic polypeptides and proteins, synthetic or natural: polymers and copolymers containing glutamic acid, aspartic acid, lysine, arginine, ornitine, other nonprotein amino acids with ionizable function in the side chain; proteins with high or low isoelectric points, such as cytochrome C, histone, protamine, trypsin, and partially hydrolyzed collagens.

Nucleic acids, oligo- and polynucleotides, and their derivatives.

Polymeric carboxylic acids: polymers and copolymers containing units of acrylic acid, methacrylic acid, maleic acid, propargylic acid, styrenecarboxylic acid, or other alkenyl- or alkenylarylcarboxylic acid; polymers and copolymers containing ionizable carboxyls in side groups on a polyamide, polyether, polyester, or polycyclic backbone.

Polymers with phosphate groups in the polymer backbone, such as polyphosphates, or in side chains, such as polyvinylphosphate.

Polymers bearing sulfo groups, such as: polyvinylsulfate, polyvinylsulfonate, polystyrenesulfonate, sulfated rosin gum (naphtenate).

Polymeric amines and amino containing heterocycles, whether in side groups or in the polymer backbone, such as: polyvinylamines, polyallylamines, polyvinylalkylamines and polyvilyltrialkylammonium salts, polyvinylpyridines, quaternized polyvinylpyridines, poly(alkylene imines), quaternized poly(alkylene imines), poly(aminoalkyl) acrylates, poly(alkylaminoalkyl) acrylates, poly(aminoalkyl) vinyl alcohols, and copolymers containing the units of the above polymers.

Polymers containing thiocarboxylic, dithiocarboxylic, thiosulfate, and thiophosphate functions in side chains or in the main polymer backbone.

The polyelectrolytes, according to the invention, may be, for example, polymeric carboxylic acid, such as polyacrylic acid, or polysaccharides, such as chondroitin sulfate A or dextran sulfate. These compounds are polymers that are soluble in water, and in such solution they acquire multiple ionic charges of the negative sign. It is understood that the present invention is not limited to the above illustrative compounds.

The coating of magnetic substance with the polyelectrolyte is achieved by a number of ways. The magnetic substance, e.g., in the form of a nanoparticle, can be formed in the presence of a polyelectrolyte, as described, for example, in U.S. Pat. No. 5,411,730, supra. Alternatively, a pre-formed magnetic substance, can be incubated with polyelectrolyte under the conditions promoting adsorption and/or chemical attachment of the polyelectrolyte to the substance. For example, a nanoparticular magnetic substance can be combined with a polyelectrolyte in an aqueous medium, preferably at low ionic strength, and treated with untrasound. For a covalent attachment (chemisorption), a polyelectrolyte containing functionalities chemically reactive with the magnetic substance can be used; for example, a sulfhydryl (—SH)-group can be introduced into the structure of the polyelecrolyte and used to react with the surface of a metal oxide magnetic nanoparticle to effect attachment of the polyelectrolyte to the nanoparticle. The polyelectrolyte is typically added in excess of the magnetic substance to achieve more complete coating of the magnetic substance. Then, the polyelectrolyte-coated magnetic substance can be optionally separated from excess polyelectrolyte, e.g., by precipitation, magnetic separation, ultrafiltration, dialysis, or size-exclusion chromatography. Surprisingly, the separation of excess polyelectrolyte did not preclude the interaction of the organic active principle with the magnetic substance to form a water-dispersible microparticle while minimizing the polyelectrolyte content in the microparticle.

The organic substance component of the microparticle is, for example, a pharmaceutical substance, that is, a substance useful in diagnosis, treatment, or prevention of a disease in a human or an animal. Typically, the pharmaceutical substance is an ionizable organic substance, that is, is capable of attaining an overall positive or negative ionic charge in aqueous, or other suitable liquid medium in which the interaction between the substance and the polyelectrolyte-coated magnetic particle takes place. The organic compound is typically a non-polymeric compound (sometimes termed as a “small molecule”), as opposed to the polyelectrolyte that coats the magnetic substance component of the microparticle, which is a polymer and comprises a chain of repeating units. In one embodiment, the organic substance is water-soluble, e.g., has solubility in water or more than 5 mg/mL. In another embodiment, the organic substance is poorly soluble in water, having aqueous solubility of less than 5 mg/mL, less than 1 mg/mL, or less than 0.1 mg/mL. It was surprisingly found that even though the organic substance within the microparticle is poorly water-soluble, the microparticle is dispersible in water, that is, stays suspended in solution for a time of one hour or more, or at least 20 hours, sufficient to administer, e.g., a pharmaceutical-containing microparticle, to a patient.

In general, any pharmaceutical which combines lipophilic properties, in particular, those resulting in low water solubility, with the presence of ionizable groups in their molecular structure, are within the scope of the invention. Examples of such ionizable groups are: amino, amidino, guanidino, azo, nitrogen-containing heterocyclic, phenolic, thiol, thiophenolic, carboxylic, 1,2-unsaturated alcohol (enol), thiocarboxylic, dithiocarboxylic, sulfo-, sulfonic, sulfinic, thiosulfonic, phosphine, phosphate, phosphonic, phosphinic, thiophosphonic and thiophosphate groups. Compounds with lipophilic properties are, for example, those containing aromatic, condensed aromatic, alicyclic, medium- and long chain aliphatic groups, or combinations thereof.

For example, such pharmaceutical may be anticancer agent doxorubicin, or dexinuguldipine, a drug known to increase the sensitivity of drug-resistant cancer cells to anticancer chemotherapy (Hoffman et. al., Biochem Pharmacol. 49, 603, 1995). Another example of such insoluble pharmaceutical is clofazimine, an antimycobacterial agent. Yet another example is amphotericin B, an antifungal agent. (Physician's Desk Reference, 1995). These pharmaceutical agents posses ionizable groups that vest into them the ability to undergo ionic dissociation and form ionic charges of the positive sign in an aqueous environment.

In another embodiment, the pharmaceutical substance is an anticancer entity. A partial listing of some of the commonly known commercially approved (or in active development) antineoplastic agents by classification is as follows. Structure-Based Classes: Fluoropyrimidines—5-FU, Fluorodeoxyuridine, Ftorafur, 5′-deoxyfluorouridine, UFT, S-1 Capecitabine; pyrimidine Nucleosides—Deoxycytidine, Cytosine Arabinoside, 5-Azacytosine, Gemcitabine, 5-Azacytosine-Arabinoside; Purines—6-Mercaptopurine, Thioguanine, Azathioprine, Allopurinol, Cladribine, Fludarabine, Pentostatin, 2-Chloro Adenosine; Platinum Analogues—Cisplatin, Carboplatin, Oxaliplatin, Tetraplatin, Platinum-DACH, Ormaplatin, CI-973, JM-216; Anthracyclines/Anthracenediones—Doxorubicin, Daunorubicin, Epirubicin, Idarubicin, Mitoxantrone; Epipodophyllotoxins—Etoposide, Teniposide; Camptothecins—Irinotecan, Topotecan, Lurtotecan, Silatecan, 9-Amino Camptothecin, 10,11-Methylenedioxy Camptothecin, 9-Nitro Camptothecin, TAS 103, 7-(4-methyl-piperazino-methylene)-10,11-ethylenedioxy-20(S)-camptothecin, 7-(2-N-isopropylamino)ethyl)-20(S)-camptothecin; Hormones and Hormonal Analogues—Diethylstilbestrol, Tamoxifen, Toremefine, Tolmudex, Thymitaq, Flutamide, Bicalutamide, Finasteride, Estradiol, Trioxifene, Droloxifene, Medroxyprogesterone Acetate, Megesterol Acetate, Aminoglutethimide, Testolactone and others; Enzymes, Proteins and Antibodies—Asparaginase, Interleukins, Interferons, Leuprolide, Pegaspargase, and others; Vinca Alkaloids—Vincristine, Vinblastine, Vinorelbine, Vindesine; Taxanes—Paclitaxel, Docetaxel. Mechanism-Based Classes: Antihormonals—See classification for Hormones and Hormonal Analogues, Anastrozole; Antifolates—Methotrexate, Aminopterin, Trimetrexate, Trimethoprim, Pyritrexim, Pyrimethamine, Edatrexate, MDAM; Antimicrotubule Agents—Taxanes and Vinca Alkaloids; Alkylating Agents (Classical and Non-Classical)—Nitrogen Mustards (Mechlorethamine, Chlorambucil, Melphalan, Uracil Mustard), Oxazaphosphorines (Ifosfamide, Cyclophosphamide, Perfosfamide, Trophosphamide), Alkylsulfonates (Busulfan), Nitrosoureas (Carmustine, Lomustine, Streptozocin), Thiotepa, Dacarbazine and others; Antimetabolites—Purines, pyrimidines and nucleosides, listed above; Antibiotics—Anthracyclines/Anthracenediones, Bleomycin, Dactinomycin, Mitomycin, Plicamycin, Pentostatin, Streptozocin; topoisomerase Inhibitors—Camptothecins (Topo I), Epipodophyllotoxins, m-AMSA, Ellipticines (Topo II); Antivirals—AZT, Zalcitabine, Gemcitabine, Didanosine, and others; Miscellaneous Cytotoxic Agents—Hydroxyurea, Mitotane, Fusion Toxins, PZA, Bryostatin, Retinoids, Butyric Acid and derivatives, Pentosan, Fumagillin, and others.

In addition to the above, an anticancer entity include without any limitation, any topoisomerase inhibitor, vinca alkaloid, e.g., vincristine, vinblastine, vinorelbine, vinflunine, and vinpocetine, microtubule depolymerizing or destabilizing agent, microtubule stabilizing agent, e.g., taxane, aminoalkyl or aminoacyl analog of paclitaxel or docetaxel, e.g., 2′-[3-(N,N-Diethylamino)propionyl]paclitaxel, 7-(N,N-Dimethylglycyl)paclitaxel, and 7-L-alanylpaclitaxel, alkylating agent, receptor-binding agent, tyrosine kinase inhibitor, phosphatase inhibitor, cycline dependent kinase inhibitor, enzyme inhibitor, aurora kinase inhibitor, nucleotide, polynucleotide, and farnesyltransferase inhibitor.

In another embodiment, the entity contained in the magnetic microparticle composition of the present invention is a therapeutic agent of anthracycline compounds or derivatives, camptothecine compounds or derivatives, ellipticine compounds or derivatives, vinca alkaloinds or derivatives, wortmannin, its analogs and derivatives, or pyrazolopyrimidine compounds with the aurora kinase inhibiting properties.

In yet another embodiment, the entity contained in the liposome composition of the present invention is an anthracycline drug, doxorubicin, daunorubicin, mitomycin C, epirubicin, pirarubicin, rubidomycin, carcinomycin, N-acetyladriamycin, rubidazone, 5-imidodaunomycin, N-acetyldaunomycine, daunoryline, mitoxanthrone; a camptothecin compound, camptothecin, 9-aminocamptothecin, 7-ethylcamptothecin, 10-hydroxycamptothecin, 9-nitrocamptothecin, 10,11-methylenedioxycamptothecin, 9-amino-10,11-methylenedioxycamptothecin, 9-chloro-10,11-methylenedioxycamptothecin, irinotecan, topotecan, lurtotecan, silatecan, (7-(4-methylpiperazinomethylene)-10,11-ethylenedioxy-20(S)-camptothecin, 7-(4-methylpiperazinomethylene)-10,11-methylenedioxy-20(S)-camptothecin, 7-(2-N-isopropylamino)ethyl)-(20S)-camptothecin; an ellipticine compound, ellipticine, 6-3-aminopropyl-ellipticine, 2-diethylaminoethyl-ellipticinium and salts thereof, datelliptium, retelliptine.

In yet another embodiment, the pharmaceutical compound contained in the magnetic microparticle of the present invention includes, without limitation, any of the following: antihistamine ethylenediamine derivatives (bromphenifamine, diphenhydramine); Anti-protozoal: quinolones (iodoquinol); amidines (pentamidine); antihelmintics (pyrantel); anti-schistosomal drugs (oxaminiquine); antifungal triazole derivatives (fliconazole, itraconazole, ketoconazole, miconazole); antimicrobial cephalosporins (cefazolin, cefonicid, cefotaxime, ceftazimide, cefuoxime); antimicrobial beta-lactam derivatives (aztreopam, cefmetazole, cefoxitin); antimicrobials of erythromycine group (erythromycin, azithromycin, clarithromycin, oleandomycin); penicillins (benzylpenicillin, phenoxymethylpenicillin, cloxacillin, methicillin, nafcillin, oxacillin, carbenicillin); tetracyclines; other antimicrobial antibiotics, novobiocin, spectinomycin, vancomycin; antimycobacterial drugs: aminosalicyclc acid, capreomycin, ethambutol, isoniazid, pyrazinamide, rifabutin, rifampin, clofazime; antiviral adamantanes: amantadine, rimantadine; quinidine derivatives: chloroquine, hydroxychloroquine, promaquine, qionone; antimicrobial qionolones: ciprofloxacin, enoxacin, lomefloxacin, nalidixic acid, norfloxacin, ofloxacin; sulfonamides; urinary tract antimicrobials: methenamine, nitrofurantoin, trimetoprim; nitroimidazoles: metronidazole; cholinergic quaternary ammonium compounds (ambethinium, neostigmine, physostigmine); anti-Alzheimer aminoacridines (tacrine); anti-Parkinsonal drugs (benztropine, biperiden, procyclidine, trihexylhenidyl); anti-muscarinic agents (atropine, hyoscyamine, scopolamine, propantheline); adrenergic dopamines (albuterol, dobutamine, ephedrine, epinephrine, norepinephrine, isoproterenol, metaproperenol, salmetrol, terbutaline); ergotamine derivatives; myorelaxants or curane series; central action myorelaxants; baclophen, cyclobenzepine, dentrolene; nicotine; beta-adrenoblockers (acebutil, amiodarone); benzodiazepines (ditiazem); antiarrhythmic drugs (diisopyramide, encaidine, local anesthetic series—procaine, procainamide, lidocaine, flecaimide), quinidine; ACE inhibitors: captopril, enelaprilat, fosinoprol, quinapril, ramipril; antilipidemics: fluvastatin, gemfibrosil, HMG-coA inhibitors (pravastatin); hypotensive drugs: clonidine, guanabenz, prazocin, guanethidine, granadril, hydralazine; and non-coronary vasodilators: dipyridamole.

In yet another embodiment, the pharmaceutical entity contained in a magnetic microparticle is poorly or sparingly soluble in water, and also possesses ionizable functional groups, as described above. Following is a list exemplifying pharmaceuticals which are poorly soluble in water at appropriate pH (6-8) and have ionizable groups in their molecular structure according to American Hospital Formulary Service, Drug Information, 1996 edition, and British Pharmacopoeia 1993-1996. This list does not include all investigational drugs and drugs not approved for use in the U.S. as of 1996 or later, however, those with similar properties with regard to lipophilicity and the presence of ionizable groups are considered within the scope of the present invention.

Antihistamines: loratadine, terfenadine, famotidine, cyproheptadine,

buclizine, cinnarizine. Amebicides: iodoquinol, mebendazole, thiabendazole, oxamniquine, timidazole. Antifungal: amphotericin B, imidazole derivatives (butoconazole, clotrimazole, econazole, itraconazole, ketoconazole, miconazole, oxiconazole, terconazole), gentian violet, nafbifine, terbinafine, clioquinol. Anti-mycobacterial: rifabutin, clofazimine. Anti-malarial: pyrimethamine, sulfadoxine. Antimicrobial quinolones: nalidixic acid. Antimicrobial sulfonamides: sulfadiazine, sulfamethazole, sulfamethoxazole, sulfalazine, sulfaxazole, sulfadimidine, sulfafurazole, silfasomidine. Anti-viral: saquinavir, ritonavir, indinavir, idoxuridine. Antineoplastic: melphalan, mercaptopurine, thioguanine. Adrenergic: salmeterol. Anticoagulants: dicumarol, nicoumalone. Antiarrhythmic: disopyramide. Dihydropyridines (Calcium channel blockers): nicardipine, nifedipine, nimodipine, felodipine, niguldipine, and their dex-enantiomers. Anti-hypertensive: reserpine and its derivatives, pindolol, prazolin. Antilipidemic: fluvastatin, gemfibrozil, pravastatin. Non-steroid anti-inflammatory: salsalate, etodolac, ibuprophen, indomethacin, ketoprophen, mephenamic acid, piroxicam, naproxen, azapropazone. Anxiolytic: benzodiazepines (clonazepam, bromazepam, alprazolam, estazolam, lorazepam, oxazepam, quazepam). Antipsychotic: haloperidol, primozide, droperidol, fluphenazine, sulpiride, perphenazine, flupenthixol. Diuretic: thiasides (bendroflumethazide, chlorotiazide, hydrochlorothiazide, hydroflumethiazide, polythiazide), metholazone, furosemide, pteridines (triamterene). Hypoglycaemic sulfonureas: glipizide, tolazamide, gliclazide, glibenclamide. Oxytocics: prostaglandines (dinoprostone). Antiprutitics and alangesics: dibucaine, phenazopyridine. Retinolc acid derivatives: tretinoin, isotretinoin. Piperidinopyrimidine vasodilators/hair growth stimulants: minoxidil. Vitamins: riboflavin, folic acid. Ovulation inducers: clomiphene. Antipsoriatic: dithranol. Antiemetic: domperidone, cyclizine. Antiestrogens: tamoxifen. Platelet aggregation inhibitors: ticlodipine, dipyridamole. Hypnotic: zopiclone, metaqualone. Drugs for treatment of peptic ulcer: omeprazole, sulfazalazine. Antidiarrheal: dipenoxylate. Anti-gout and anti-thyroid: allopurinol, propylthiouracil. Immunosupressant: azathioprine. Steroids: hydrocortizone hydrogen succinate, stanozolol. Cough supressant: noscapine. Anorexogenic: dexfenfluramine.

In addition, the inventive material may include organic substances having non-pharmaceutical utility, such as, for example: organic dyes and pigments used in fabric dyeing, inks, paints, as fillers for colored plastics, etc.; volume-increasing, gas producing substances, used in production of foams and in food industry, and in other applications where stable, in particular, aqueous-based, organic colloids with magnetic properties are desirable. Any type of natural or synthetic organic dyes (water-soluble) or pigments (less water-soluble) can be used as the organic substance component of the microparticles, as long as their molecular structure affords binding to the magnetic colloidal particles, for example, because of ionization to the ionic charge opposite to that of the polyelectrolyte coating of the magnetic colloid, as described below. Based on these criteria, appropriate dye and pigment structures would be routinely selected by persons skilled in the art of chemistry.

The method of manufacturing of the microparticles according to the invention comprises contacting the magnetic substance, e.g. in a colloidal or nanoparticle form, coated with a polyelectrolyte of a non-zero net charge, with an organic compound, e.g., drug, in a solution, where the organic compound is chosen so that under the contacting conditions its molecules are capable of attaining the ionic charge opposite to that of the polyelectrolyte coating. For example, if the polyelectrolyte is a polyanion (negatively charged), and contains acidic functional groups, such as, carboxylate, phosphate, sulfate, or borate, the organic substance would typically contain cationically dissociating functional groups, such as amine or heterocyclic nitrogen moieties. If the polyelectrolyte is a polycation (positively charged), for example, a polymeric amine or ammonium, the organic substance would typically contain acidic groups, such as, for example, carboxylate, phosphate, sulfate, or borate.

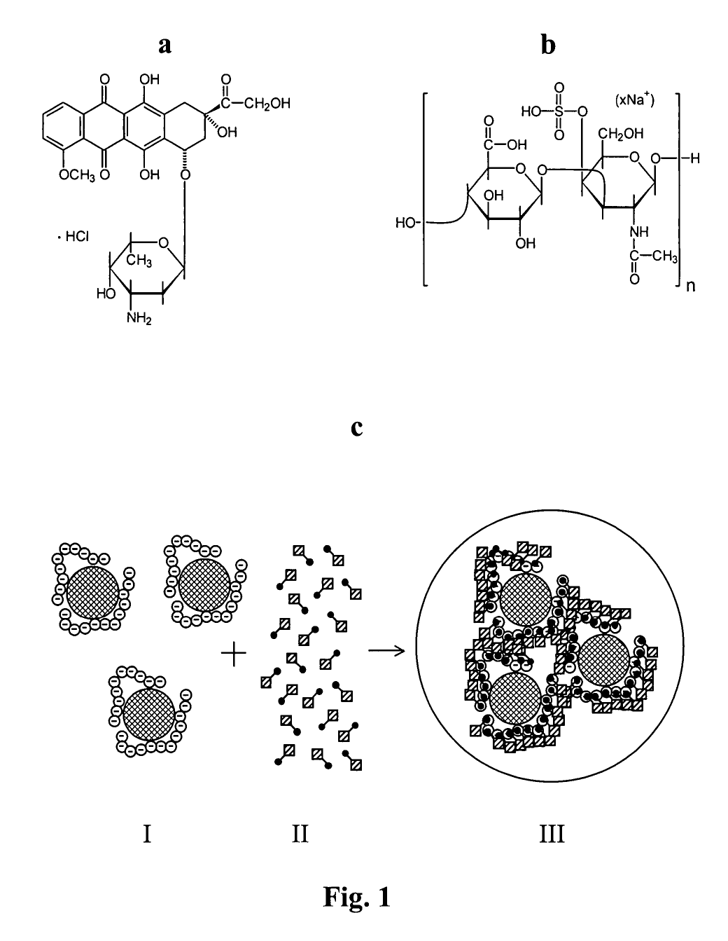

The above method, and the structure of the above described material, are schematically illustrated on FIG. 1 by the example of the drug Doxorubicin and the polyelectrolyte Chondroitin Sulfate A.

The polyelectrolyte-coated magnetic substance, e.g., in the form of a nanoparticle, is contacted with the pharmaceutical substance to produce magnetic microparticle containing said pharmaceutical substance. The contacting step is performed, e.g., simply by mixing of the solutions containing the organic substance and the polyelectrolyte-coated magnetic substance in aqueous medium, such as water, buffer solution, or physiologically acceptable (e.g., injectable) solution.

While relatively water-soluble organic active principles are added to the polyelectrolyte-coated magnetic substances typically in the from of aqueous solutions, we have surprisingly discovered that poorly water-soluble, ionizable organic compounds can be incorporated into microparticles along with the magnetic substance if they are added to an aqueous solution of polyelectrolyte-coated magnetic substance in the form of a solution in a water-miscible organic solvent according to the procedure described in U.S. Pat. No. 6,048,650, incorporated herein by reference. While U.S. Pat. No. 6,048,650 teaches that hydrophilic microparticles of water-insoluble, ionizable organic compounds form when mixed with polyelectrolytes in aqueous solution, we unexpectedly found that polyelectrolyte-bound magnetic substances, such as, e.g., superparamagnetic nanoparticles, similarly effect the formation of hydrophilic, water-dispersible microparticles incorporating both the organic compound and the magnetic substance, with high efficiency (70% or more, or even 90% or more) for entrapment of both components.

Following the mixing step, if organic solvent is present, it can be optionally removed by dialysis, magnetic separation, ultrafiltration, size-exclusion chromatography, or evaporation, including lyophilization. Lyophilization of the microparticle preparation can be performed in the presence of a stabilizer, such as carbohydrate, e.g., sucrose, lactose, trehalose, or a neutral hydrophilic polymer, such as polyethyleneglycol, polyvinylpyrrolidone, polyacrylamide, or dextran. Magnetically separated or lyophilized microparticles can be resuspended (reconstituted) in any appropriate, e.g., aqueous, medium for storage and/or administration to a patient, when the microparticle comprises a pharmaceutical compound.

The size of magnetic microparticles is typically in the range allowing passage through the blood capillaries (less than 6 micrometers in diameter), more preferably less than 2 micrometers, and most preferably lies between about 50 nanometers and 1 micrometer. To obtain the desired size, the microparticle suspension can be fractionated by passage through the sieves or filters with defined pore size, such as polycarbonate or polyester track-etched membrane filters.

The microparticles typically contain more than 50% of the organic substance and the magnetic component taken together, and are hydrophilic, that is, form dispersions in water stable against precipitation and/or aggregation for a period of time typically at least 1 hour, more preferably, at least 6 hours, or most preferably 20 hours or more. The ratio of magnetic substance to the pharmaceutical compound may vary according to the particular tasks, but typically would be in the range of 5-95% of magnetic substance, more often in the range 10-90% of magnetic substance, and most often in the range of 20-80% of the magnetic substance, counting on the sum of the magnetic substance and the pharmaceutical compound.

Magnetic microparticles of the invention have many utilities. One is magnetically directed targeting, where the particles are confined or retained in the desired area by a magnetic field. Another is a combination of pharmaceutical action and remote heating of the magnetic component achieved by the application of a high frequency oscillating electromagnetic field. The release of heat may trigger or modulate the release of a pharmaceutical substance, therefore providing a valuable tool to control the activity of the pharmaceutical. To achieve such triggered or modulated release, the polyelectrolyte component is, for example, a polymer with the cloud point (coil-globule transition) in the desired temperature range, such as a derivative of poly-N-isopropylacrylamide or co-polymer of N-isopropylacrylamide and acrylic or metacrylic acid. Another utility is the possibility to non-invasively detect and monitor the distribution of the magnetic microparticles, e.g., in the patient's body using magnetic resonance based methods, such as magnetic resonance imaging. Magnetic microparticles of the invention comprising dyes or pigments instead of pharmaceutical compounds have utility as components of magnetically readable and magnetically dischargeable inks.

It is recognized that the magnetic pharmaceutical microparticles of this invention may contain more than one kind of magnetic material, or of the drug, or even of the polyelectrolyte coating, and may contain other useful additives.

Particularly useful additives are circulation effectors and targeting ligands.

Circulation effectors prolong the persistence of microparticles in the bloodstream, this increasing their accumulation in the diseased areas of the body, such as tumors. See, for example, U.S. Pat. No. 5,013,556; U.S. Pat. No. 5,213,804. One class of circulation effectors are hydrophilic polymers, such as poly(ethylene glycol), polyacrylamide, polyoxazolidine, terminally grafted on the microparticle surface. To achieve grafting, the circulation-effecting polymer can be made, for example, with a terminal functional group reactive with the magnetic substance, the pharmaceutical substance, or the polyelectrolyte component of the microparticle, and allowed to react with the microparticle to effect covalent attachment. Alternatively, an “anchor” group, such as a lipid group or a polyelectrolyte chain electrostatically attracted to the pharmaceutical, magnetic, or polyelectrolyte component of the particle may be added before or after the microparticle formation. Typically, the polymer with molecular weight 500-20,000, or preferably, as in the case of poly(ethylene glycol), molecular weight 500-5,000 is used in the amount from 0.1% to 20%, more preferably from 0.5% to 10% of the particle mass.

Targeting ligands are substances that specifically bind to the cells or areas of the body that bear characteristic molecular markers of the disease. The utility of targeting ligands to deliver microparticulate pharmaceuticals is well known. See, for example, U.S. Pat. No. 5,213,804. Ligand is a member of a specific binding pair, wherein a second member is present on the exposed area of the target cell or tissue. Such binding pairs are, for example, antibody-antigen, effector-receptor, enzyme-substrate, and lectin-carbohydrate. Preferred types of targeting ligands are antibodies, that is, polypeptides comprising complementarity-determining regions (CDRs) of an immunoglobulin. It is understood that whole immunoglobulins, as well as their antigen-binding fragments, whether naturally or recombinantly derived, such as Fab′, Fv, single chain antibodies, and single domain antibodies, are included. Artificial peptides, such as derived through selection of biodisplay libraries, e.g., phage display libraries, as well as natural peptides (hormones) binding various cell surface receptors, are suitable as ligands. Nucleic acids and small organic molecules selected for specific binding to target tissues or cells are also suitable ligands. In some cases, it is desirable to have a ligand that causes internalization of the microparticle into a target cell, as taught, for example, in U.S. Pat. No. 6,214,388.

Targeting ligands can be attached to the surface of microparticles by a variety of conjugation methods known in the art. See Hermanson, Bioconjugate Techniques, Academic Press, 1996. The attachment can be direct to the particle surface or via a spacer group, such as a hydrophilic polymer spacer, similar to the circulation effector polymer. It this case, the ligand can be attached to a free distal terminus of the microparticle-grafted polymer spacer via a chemically active group, or pre-attached to a polymer-anchor construct, and such ligand-polymer-anchor construct can be incorporated into the microparticle by addition to the magnetic substance-polyelectrolyte-pharmaceutical compound combination before, or after the particle formation.

The microparticles may be formulated for the medical use by any appropriate, known method, such as, for example, dilution into a physiologically acceptable injection medium, sterilization, further formulation and combination with various other medicaments, and that these microparticles may be administered to the body by any known method, as well as used extracorporeally (“ex vivo”). The microparticles can be administered as components of any desirable desirable dosage forms, such as, tablets, patches, gels, cremes, ointments, aerosol strays, and the like. The microparticle-containing compositions can be administered orally or via any suitable parenteral route according to the physician's task. It is also recognized that, although in the present disclosure we describe a formulation of a pharmaceutical substance into magnetic microsphere, the same formulation can be performed with any other useful substance having appropriate molecular characteristics, and the resulting magnetic microparticles may have utility in the fields other than medicine, such as, for example, biotechnology, enzyme immobilization, manufacturing of various materials, analytical chemistry, and such utility is also covered by the present disclosure.

### EXAMPLES

### Example 1

### Magnetic Iron Oxide Colloid Coated with Chondoitin sulfate A

50 mg of chondroitin sulfate A were dissolved in 1 mL of distilled water. To this solution 0.3 mL of colloidal gamma-ferric oxide containing 68.4 mg/mL iron were added, and the mixture was treated with ultrasound for 15 min. Then 0.1 mL of a solution containing 1.5 M NaCl and 0.2 M Hydroxyethylpiperazinesulfonate-Na at pH 7.4 (10×HEPES-NS) was added, and the mixture was chromatographed on Sepharose CL-4B using 0.15 M NaCl-20 mM HEPES-Na, pH 7.4, as eluent. The dark-colored fraction containing ferrocolloid was collected and sterilized by filtration through a 0.22 μm filter. The product has 5.6 mg/mL of iron and 0.96 mg/mL of chondroitin sulfate A. FIG. 3 shows size distribution histogram obtained from the transmission electron microscopy view of the colloidal particles produced according to this example.

### Example 2

### Magnetic Iron Oxide Colloid Coated with Polyethyleneimine

0.3 mL of colloidal gamma-ferric oxide containing 68.4 mg/mL iron were added to 1 mL of water solution containing 44 mg of polyethyleneimine (Polymine P, Sigma Chemical Co.) adjusted to pH 3.5 with hydrochloric acid. The mixture was treated with ultrasound for 15 min. The resulting solution was chromatographed on Sepharose CL-4B using deionized water as eluent and sterilized by filtration through a 0.22 μm filter to give polyethyleneimine-coated ferrocolloid, with iron concentration of 1.54 mg/ml.

### Example 3

### Magnetic Doxorubicin

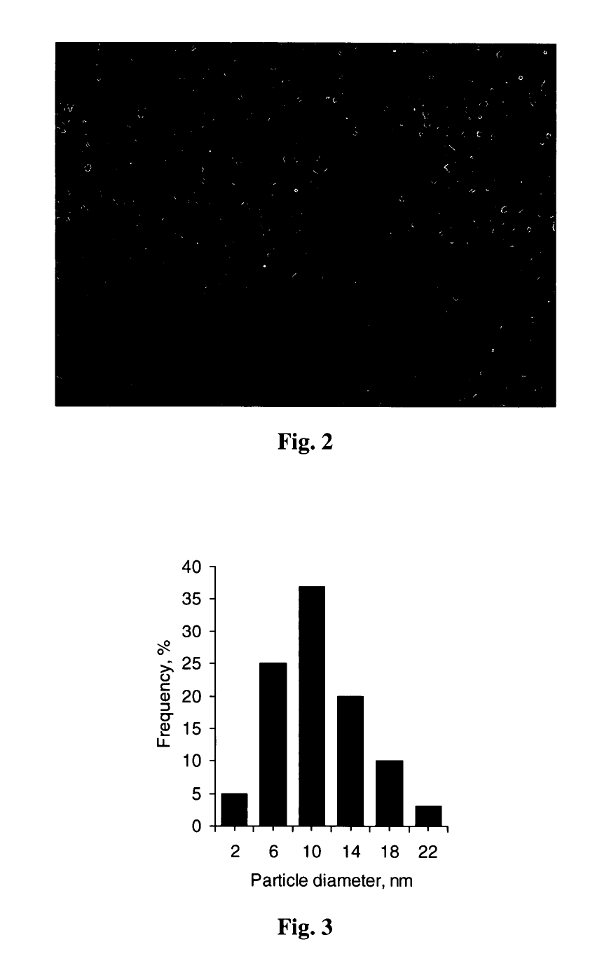

20 μL of the chondroitin sulfate-coated ferric oxide obtained according to the Example 1 and 30 μL of doxorubicin injection solution USP (2 mg/mL of doxorubicin) were mixed in total volume of 100 μL of aqueous solution also containing 0.15 M NaCl and 20 mM HEPES-Na, pH 7.4 (1×HEPES-NS). The microspheres formed immediately. They were separated by exposure of the reaction vessel to a magnet and resuspended by vortexing in a suitable amount of 1×HEPES-NS. The microspheres had 74.9% of Fe₂O₃, 16.0% of doxorubicin, the rest chondroitin sulfate (percentages are of total dry mass). FIG. 2 shows a fluorescent microscopy image of magnetic doxorubicin produced according to this example. The mouse red blood cells (larger round bodies approx. 6 nm in diameter) are added for comparative evaluation of particle size.

### Example 4

### Magnetic Doxorubicin—Another Example

Procedure of Example 3 was followed, but the volume of doxorubicin solution was reduced to 20 μL. The microspheres had 77.6% of Fe₂O₃, 13.0% of doxorubicin, the rest chondroitin sulfate (percentages are of total dry mass).

### Example 5

### Magnetic Dexniguldipine

20 μL of chondroitin sulfate-coated ferrocolloid obtained according to Example 1 were mixed by vortexing with 40 μL of a solution containing 2 mg/mL of dexniguldipine (BYK Gulden Pharmaceuticals) and 16% DMSO. Water and 10×HEPES-NS were also added to achieve final volume of 100 μL, 0.15 M NaCl and 20 mM HEPES. After 2 hours the microspheres were separated by a magnet and resuspended in a suitable volume of 1×HEPES-NS. The microspheres contained (of dry mass): dexniguldipine 46.1%, Fe₂O₃ 48.3%, balance chondroitin sulfate.

### Example 6

### Magnetic Methotrexate

40 μL of polyethyleneimine-coated ferrocolloid obtained as in Example 2 was mixed with 30 μL of methotrexate (Ametopterin, Sigma Chemical Co.) solution (4 mg/mL in water, pH 7.4). 10 μL of water and 10 μL of 10×HEPES-NS. The microspheres were separated by a magnet and resuspended in 1×HEPES-NS. The microspheres contained 86.9 mg Fe₂O₃, and 81.3 mg methotrexate.

### Example 7

### Biological Activity of Magnetic Doxorubicin

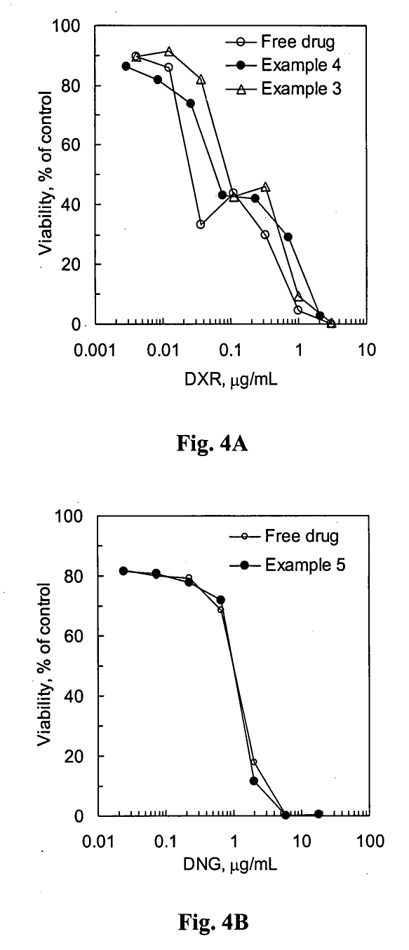

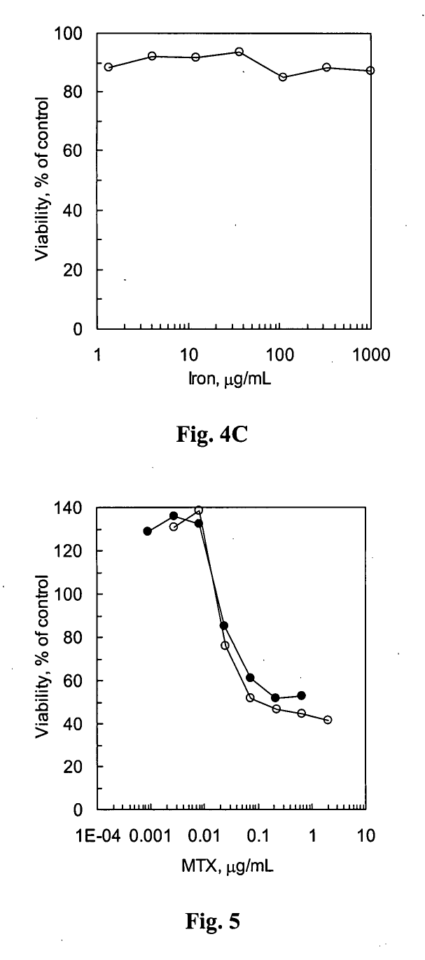

Human epidermoid carcinoma cells (KB) were grown in RPMI 1470 medium supplemented with 10% fetal calf serum at 37° C. and 5% CO₂. The cells were plated in a 96-well tissue culture plates at the density of 5,000/well and let to attach and acclimate for 48 hours. Free doxorubicin and resuspended magnetic doxorubicin microspheres obtained according to Examples 3 and 4 above were added to the wells to cover the concentration range of 0.01-10 microgram of drug/ml. After 19 hour exposure to the drug, cells were exposed to fresh medium for 72 hours, and the cell viability was analyzed using conventional tetrasolium (MTT) assay as described in the literature. As shown on FIG. 4A, the cytotoxicity of the magnetic drug was close to that of the free drug. The chondroitin sulphate coated ferric oxide alone was non-toxic to the cells, as shown on FIG. 4C.

### Example 8

### Biological Activity of Magnetic Dexniguldipine

Cells were grown and plated as described in Example 7. Free dexniguldipine (solution) or magnetic dexniguldipine obtained as in Example 5 was added to the cells to cover the range of dexniguldipine concentrations of 0.03-15 microgram/ml. Cell viability assay and other assay conditions were as in Example 7. As shown on FIG. 4B, the cytotoxicity of magnetic dexniguldipine is the same as of the free drug.

### Example 9

### Biological Activity of Magnetic Methotrexate

Human small cell lung carcinoma cells (NCI-H1048) were prepared for assay as described in the Example 7 above. The cells were exposed to the various concentrations (0.001-2 microgram/ml) of methotrexate in the form of drug solution or in the magnetic form obtained as described in the Example 6 above for 94 hours. The cell viability after drug exposure was determined using MTT assay. The results, displayed on FIG. 5, show that the cytostatic properties of magnetic drug are close to these of the free drug.

### Example 10

### Maghemite Coated with Dextran Sulfate

Gamma-ferric oxide (maghemite) was obtained by drying of the colloidal material obtained by ammonia precipitation from aqueous solution containing equimolar amount of ferric and ferrous chloride with aqueous ammonia and air oxidation. 104.1 mg of maghemite was dispersed in the solution of 101.3 mg dextran sulfate (DS, Sigma Chemical Co. product No. D5224) in 3 mL of water using trituration w/glass rod, and then sonicated using tapered probe for 10 min, using Branson Sonifier 250 with power setting at 20-22 on the scale. The reaction mixture was magnetically separated by exposure to 0.25″×0.25″ NeFeB magnet, the supernatant removed, and the pellet was redispersed in 1 mL of water by brief (1 min) sonication. The dispersion was centrifuged on Eppendorf centrifuge at 5,000 rpm 3 min, and the supernatant liquid was passed through 0.2-micrometer polyethersulfone filter. This material contained 16.8 mg/mL of iron.

### Example 11

### Magnetic Microparticles Containing Clofazimine

0.1 mL of dextran sulfate-coated maghemite preparation according to Example 10, 0.4 mL water, and 0.4 mL of 5 mg/mL solution of clofazimine (Sigma Chemical Co) in DMSO were mixed in a glass vial. As a control, 0.4 mL clofazimine solution was mixed with 0.5 mL of water without magnetic substance and/or a polyelectrolyte. To the mixture, added 0.03 mL of 5 M NaCl, and the particles were separated by a magnet, as in Example 10. The supernatant was discarded, and the magnetic pellet was redispersed in 1 mL water. Reprecipitation, magnetic separation, and redispersion steps were repeated, and the particles were redispersed in 1 mL water using brief (1 sec) sonication. This preparation contained 0.58±0.02 mg/mL of clofazimine, and 1.14±0.03 mg/mL of iron. Drug/iron ratio 0.51+/−0.03.

### Example 12

### Aggregation Stability of Dextran Sulfate-Coated Maghemite, Magnetic Clofazimine Microparticles, and Clofazimine in Water

Particle size in the preparations made according to Examples 9 and 10 were determined using quasielastic light scattering method one hour after preparation and after 20 hours storage at ambient temperature. The following size data are in nanometers, mean±SD, determined using unimodal (Gaussian) method. ND—gross precipitation; not determinable. The size of both polyelectrolyte-coated maghemite and magnetic clofazimine microparticles was stable over the period of observation.

TABLE 1

| Preparation | 1 hour | 20 hours |
|---|---|---|
| Maghemite-DS (Example 10) | 73.9 ± 31.6 | 72.6 ± 32.5 |
| Magnetic clofazimine (Example 11)o | 228.4 ± 94.9 | 220.8 ± 88.0 |
| Clofazimine in water (Example 11) | 265.5 ± 107.9 | ND |

### Example 13

### Magnetic Microparticles Containing Amphotericin B

Antifungal antibiotic Amphotericin B (AMB; obtained from Sigma Chemical Co., USA) was dissolved in 80% (vol.) aqueous dimethylsulfoxide (DMSO) at 2 mg/mL. Aliquots of chondroitin sulfate A (ChSA)-coated magnetic iron oxide colloid, containing 0.0566 mg of iron and 0.0096 mg ChSA in HEPES-NS were combined with aliquots of AMB solution, containing 0.01-0.05 mg AMB, to achieve final volume of 0.1 1-0.15 mL, and mixed by vortexing briefly. The particles were separated by exposure of the mixtures to a magnet (1.5 min), and the supernatant fluids were analyzed for iron, AMB, and ChSA, using spectrophotometric methods. The incorporation of AmB in the particles was practically complete. Based on the residual amounts of iron, AMB, and ChSA the particle compositions were determined (Table 2), wherein the totality of magnetic substance (ferric oxide) and the antibiotic (AMB) was 92.3%-93.6%, and the content of the antibiotic drug was 27%-42%, of the particle dry mass.

TABLE 2

| Iron oxide-ChSA | AMB | Particle composition | | |
|---|---|---|---|---|
| added (mg iron) | added, mg | Iron oxide, % | ChSA, % | AMB, % |
| 0.0566 | 0.01 | 65.3 | 7.7 | 27.0 |
| 0.0566 | 0.02 | 59.0 | 7.0 | 34.0 |
| 0.0566 | 0.03 | 57.6 | 6.8 | 35.6 |
| 0.0566 | 0.04 | 53.7 | 6.4 | 39.9 |
| 0.0566 | 0.05 | 51.8 | 6.2 | 42.0 |

The examples and embodiments provided herein are to illustrate the invention without limiting of its scope. Various modifications of the invention will be readily apparent to one of ordinary skill in the art and are to be included within the spirit and purview of this application and scope of the appended claims. All publications, patents, and patent applications cited herein are hereby incorporated by reference in their entirety for all purposes.

We claim:

1. A composition comprising microparticles, said microparticles comprising:
(i) a magnetic substance;
(ii) a polymer associated with said magnetic substance, said polymer having functional groups ionizable to produce a charge sign; and
(iii) an ionizable organic compound,
wherein said ionizable organic compound in aqueous medium is ionizable to the charge sign opposite to that of said polymer, and
wherein said magnetic substance and said organic compound in totality constitute at least 50% of the microparticle dry mass, and
wherein said microparticles are dispersible in water.

2. A composition of claim 1 wherein said magnetic substance and said organic compound in totality constitute at least 70% of the microparticle dry mass, or at least 90% of the microparticle dry mass.

3. A composition of claim 2 wherein said organic compound constitutes at least 10% of the microparticle dry mass.

4. A composition of claim 1 wherein said organic compound is poorly soluble in water.

5. A composition of claim 1 wherein said organic compound is a pharmaceutical, a dye, or a pigment.

6. A composition of claim 5 wherein said organic compound is an anticancer drug, an antimicrobial drug, doxorubicin, methotrexate, dexniguldipine, clofazimine, or amphotericin B.

7. A composition of claim 1 wherein said magnetic substance is ferrite, magnetite, or maghemite.

8. A composition of claim 5 wherein said organic substance is a pharmaceutical, and said microparticle comprises on its surface a coating effective to extend the microparticle blood circulation time.

9. A composition of claim 1 wherein said microparticle comprises on its surface a coating comprising a hydrophilic polymer, a poly(alkyl ether), or poly(ethylene glycol).

10. A composition of claim 5 wherein said microparticle comprises a targeting moiety.

11. A composition of claim 9 wherein said microparticle comprises a targeting moiety linked to said coating.

12. A composition of claim 1 wherein said organic substance is a pharmaceutical, and microparticles are in a pharmaceutically acceptable medium.

13. A composition of claim 1 wherein said microparticles have the average size between about 10 nanometers and about 10 micrometers, or between about 30 nanometers and about 3 micrometers.

14. A composition of claim 1 wherein said magnetic substance in the form of a ferromagnetic, paramagnetic, or superparamagnetic nanoparticle, said nanoparticle having at least one magnetic domain.

15. A composition of claim 14 wherein said nanoparticle of said magnetic substance has the size between about 1 nm and about 100 nm.

16. A method of making a microparticle-containing composition, said method comprising the step of contacting a magnetic substance dispersed in a liquid medium and having a charged polymer associated with said magnetic substance and ionized in said medium to attain a charge sign, with an ionizable organic compound attaining in said medium a charge sign opposite to that of said charged polymer, to effect formation of a microparticle.

17. A method of claim 16 wherein magnetic substance and said organic compound in totality constitute at least 50% of the microparticle dry mass.

18. A method of claim 16 wherein magnetic substance and said organic compound in totality constitute at least 70% of the microparticle dry mass, or at least 90% of the microparticle dry mass.

19. A method of claim 16 wherein said liquid medium is essentially free of said charged polymer not associated with said magnetic substance.

20. A method of claim 16 wherein said liquid medium comprises water.

21. A method of claim 20 wherein said organic substance is poorly soluble in water.

22. A method of claim 21 wherein said contacting comprises mixing of said magnetic substance dispersed in a water-containing medium with a solution of said organic substance in a medium comprising a water-miscible organic solvent.

23. A method of claim 16 wherein said organic compound is a pharmaceutical, further comprising a step of coating said microparticle with a substance effective to prolong the microparticle blood circulation time.

24. A method of claim 16 further comprising a step of coating said microparticle with a hydrophilic polymer.

25. A method of claim 24 wherein said coating comprises contacting the microparticle with the hydrophilic polymer linked to a hydrophobic moiety.

26. A method of claim 24 wherein said hydrophilic polymer further comprises a targeting moiety.

27. A method of treatment of a patient comprising administering to the patient an effective amount of the composition of claim 12.
